(19) United States
(12) Patent Application Publication
(10) Pub. No.: US 2026/0108599 A1
(43) Pub. Date: Apr. 23, 2026

(54) THERAPEUTIC COMPOUND AND METHOD OF PREPARING A THERAPEUTIC VACCINE FOR THE TREATMENT OF ALLERGIC AND AUTOIMMUNE DISEAES USING IMMUNIZATION WITH ENGINEERED BACTERIAL DNA OR CpG MOTIFS DERIVIDE FROM COMMENSAL BACTERIA SUCH AS Bifidobacterium longun subsp. infantis.

(71) Applicants: Joseph A. Bellanti, Washington, DC (US); Dongmei LI, Germantown, MD (US)

(72) Inventors: Joseph A. Bellanti, Washington, DC (US); Dongmei LI, Germantown, MD (US)

(21) Appl. No.: 18/831,298

(22) Filed: Oct. 18, 2024

(51) Int. Cl. A61K 39/385; A61K 39/00

(52) U.S. Cl. CPC A61K 39/385; A61K 2039/55561; A61K 2039/6025

(57) ABSTRACT

We disclose a biochemical compound comprising CpG moieties derived from human commensal bacteria that exhibit the ability to suppress immune system responses in patients that suffer from autoimmune and allergic diseases arising from overactive immune systems. We outline several methods for use of the compound in the treatment of individuals suffering from various types of auto-immune and related diseases.

### CROSS REFERENCE TO RELATED APPLICATIONS

This application claims the benefit of U.S. Provisional Application No. 63/629,452, filed on Oct. 27, 2023, the entirety of which is incorporated by reference herein.

### BACKGROUND

The human immune system can be understood as having three main components: the innate immune response, the adaptive immune response, and a third component that comprises tissue-damaging responses (FIG. 1). When the innate and adaptive responses operate efficiently and identify and eliminate an invading foreign substance (known as an antigen), the immune response is considered beneficial and reflects a state of immune health. However, if the immune system is deficient and struggles to handle some antigen, with the result that it persists, this will lead to the third type of response, resulting in tissue damage and inflammation under conditions such as chronic infections, allergies, autoimmune diseases, or cancer.

The innate immune system consists of various cells and substances that provide the first line of defense against foreign invaders. Specialized cells called antigen-presenting cells (APCs), including dendritic cells and macrophages, play a crucial role in processing these antigens and presenting the processed antigen to T cells in the adaptive immune system (FIG. 1), in effect profiling the antigen and then activating the T cells to become so-called effector T cells.

T cells are a type of white blood cell of the immune system and are divided into two main types: CD4+ helper T cells and CD8+ cytotoxic T cells (FIG. 2). CD4+ helper T cells are further categorized into the following CD4+ T cell subsets:
Th1 cells: Promote cellular immunity and are involved in responses to intracellular pathogens.
Th2 cells: Support humoral immunity and are involved in responses to extracellular pathogens or allergens with allergic reactions.
Th17 cells: Play a role in autoimmunity and defense against fungal and certain bacterial infections.
Treg cells (Regulatory T cells): Help regulate and suppress immune responses to maintain homeostasis and prevent autoimmunity and inflammation.
   These subsets differentiate from naïve CD4+ T cells based on the environment provided by cytokines—hormone-like substances produced by cells that help communicate and coordinate the immune response—and other signals they receive during activation (FIG. 2).

The immune system strives to maintain a balance between these proinflammatory and anti-inflammatory forces, ensuring it operates smoothly. In cases of allergies and autoimmune diseases, Treg numbers are often decreased, while in tumor immunity—where cancer cells manage to disguise themselves as harmless—tumors can paradoxically stimulate Treg activity as an evasion strategy to promote their own growth favoring tumor progression.

While the stimulation of Treg cells has potential therapeutic benefits in treating allergic and autoimmune diseases where Treg numbers are reduced, if for any reason they are further diminished there are also potential adverse effects such as worsening of the allergic or autoimmune manifestations. In contrast, excessive or non-specific Treg stimulation could suppress immune responses in ways that promote tumor growth by inhibiting anti-tumor immunity.

For the purpose of this application, we define a “vaccine” as any compound which affects a patient's immune system, with the goal of treating, curing, or preventing the onset of a specific disease. Therefore, in our terminology vaccines can be either preventative, such as those currently in use for polio, measles, or rabies- or therapeutic, and can be used as a treatment method even after a patient has already contracted the disease.

Preventive vaccines, traditionally associated with infectious diseases such as measles, mumps and rubella, are administered to individuals who are at risk of contracting a specific infectious disease but have not yet been exposed to the pathogen. Their primary aim is to establish protective immunity and thwart future infections.

Therapeutic vaccines are a more recent concept in vaccine usage, extending beyond the prevention of infectious diseases to encompass the treatment of various medical conditions, including allergic diseases, autoimmune disorders, chronic infectious diseases, hypersensitivity diseases, and cancers driven by disordered or dysfunctional immune system responses. Therapeutic vaccines, therefore, are administered to individuals who already exhibit symptoms of a particular disease or condition. Their principal objective is to treat or mitigate the effects of an existing disease, typically by modulating or reshaping the body's immune response against the underlying mechanistic cause responsible for the disorder.

Both allergic and autoimmune diseases are disorders of the immune system. The autoimmune diseases manifest either as localized or as systemic disorders of the immune system and are not caused by identifiable pathogens. Some of the most well-known of these diseases include systemic lupus erythematosus (SLE), rheumatoid arthritis (RA), type I diabetes, and multiple sclerosis. Alzheimer's disease, amyotrophic lateral sclerosis (ALS), and Parkinson's disease are yet another group of neurodegenerative disorders that are believed to have an autoimmune etiology. In contrast, allergic diseases are largely triggered by identifiable foreign substances known as allergens and encompass conditions like allergic rhinitis (hay fever), asthma, eczema, and food allergies.

The etiology of both allergic and autoimmune conditions is primarily attributed to a deficiency in suppressor T-cells, also known as Treg cells, which serve as regulators of the immune system, or to an inability to recruit these suppressor T-cells in adequate numbers or at a sufficient pace. This deficiency can, in turn, lead to an exaggerated cytokine response, promoting excessive inflammation, damaging host tissue, and giving rise to most of the prevalent characteristics of these conditions.

Pharmacological medications are available to suppress immune responses in the treatment of allergic and autoimmune conditions. For instance, steroids are widely used due to their general immunosuppressive properties. However, this approach has significant drawbacks. The long-term side effects of steroid treatment can damage other organ systems and lead to cardiovascular problems and hypertension and bone loss. In addition, steroids may damage the immune system itself, making it difficult for it to quickly recover its function if the patient encounters a new and potentially life-threatening immunological challenge during treatment.

Therefore, we propose a method for artificially suppressing an amplified or exaggerated immune response that may for some patients offer better and more adaptive control of the immune system.

CpG oligodeoxynucleotides (CpG ODNs) are short, single-stranded synthetic DNA molecules consisting of a cytosine triphosphate deoxynucleotide (‘C’) followed by a guanine triphosphate deoxynucleotide (‘G’). The ‘p’ in CpG refers to a phosphodiester link between consecutive nucleotides, although some ODNs have a modified phosphorothioate (PS) backbone instead.

A crucial aspect of the role of CpG motifs in immunology, which forms the central focus of this patent application, lies in the recent discovery that when CpG motifs remain unmethylated, they act as immunostimulants, actively enhancing the immune response. Conversely, when these CpG motifs undergo methylation, they transition into immune silencing agonists.

Recent findings from Applicants'laboratory have shown that this silencing effect is achieved through the induction of regulatory T cells (Treg cells).

### BRIEF SUMMARY

We disclose a vaccine composition that may offer patients relief from a wide variety of allergic or autoimmune or related diseases. The compound is known to promote the creation and maintenance of suppressive T-cells—also known as Treg cells—which will regulate certain important aspects of the overall immune response.

This innovative treatment regimen offers a novel form of therapy that can be administered by injection, inhalation, orally, or topically on the skin, and that can function as a therapeutic vaccine for certain patients suffering from allergic and autoimmune disorders. Since the therapy specifically targets the recruitment of Treg cells from the patient's own immune system, it circumvents many of the risks associated with conventional steroid treatments, which often lead to a reduction in both the overall capacity of the immune system as well as the robustness of its response.

### BRIEF DESCRIPTION OF THE DRAWINGS

FIG. 1 is a depiction of the three-components of the human immune system.

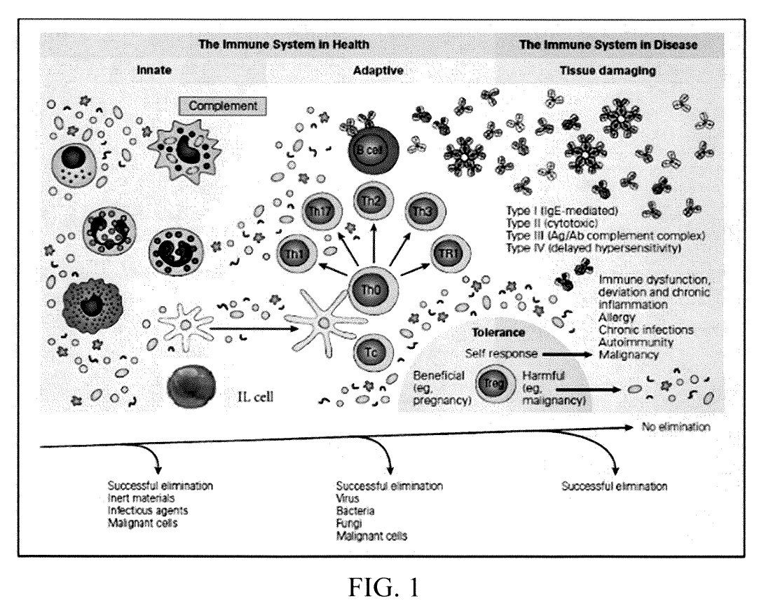

FIG. 2 is a depiction of the two main types of T cells: the CD4+ helper T cells and CD8+ cytotoxic T cells and the CD4+ T cell subsets.

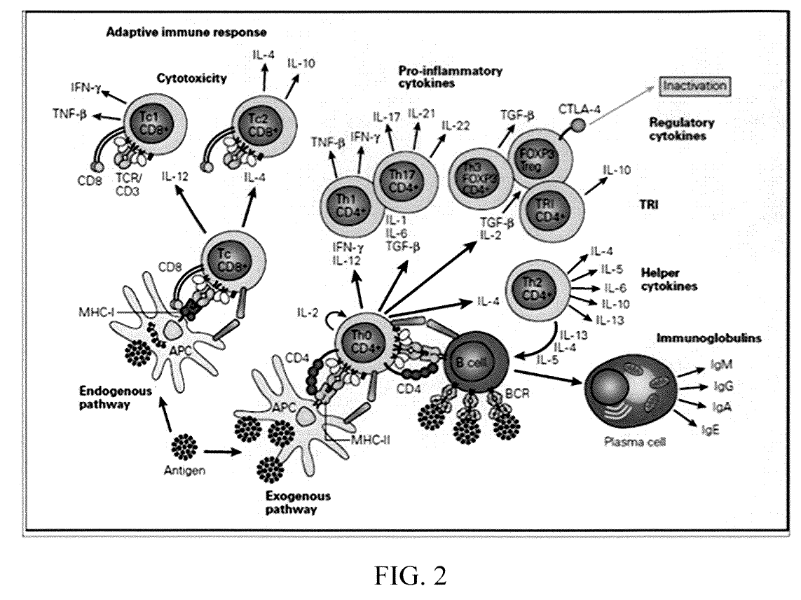

FIG. 3 is a schematic structural representation of methylated (ODNA and ODNX) and unmethylated (ODNB) nucleotide oligos derived from the genomic sequence motifs of B. longum subsp. infantis, as described in our previous study. In the ODNA molecule, “M” denotes the points of attachment of methyl groups to the cytosine residues at the 5 position of two CpG sites. “*”indicates the phosphorothioate bonds.

FIG. 4 is a schematic representation of enumeration of splenic anti-inflammatory Treg cells identified by cell-associated FOXP3+ CD25+, serum cytokine levels of IL-10 and IL-4, and mast cells in mouse skin.

### DETAILED DESCRIPTION

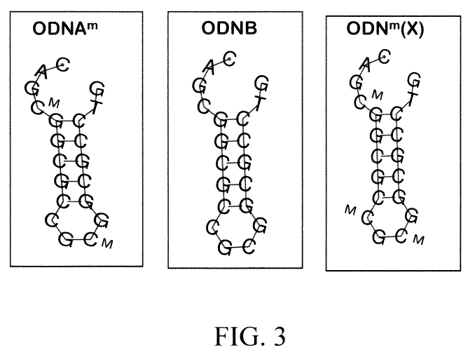

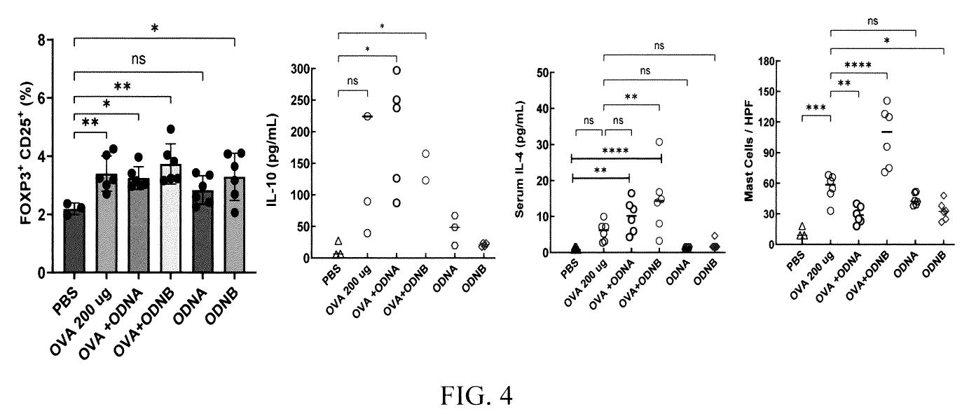

The composition of the vaccine described in this invention consists of a methylated cytosine-phosphodiester-guanine (CpG) motif (BI-T2 m5C) derived from Bifidobacterium longum subsp. infantis. The methylated motif (RGCmeGGCGCC) includes at least one occurrence of one or more of the following methylated motifs: (RGCmeGGCGCC), (RGCmeGGCmeGCC), and (RGCGGCmeGCC), schematically represented as ODNA, ODNB, or ODN(X) in the case of duplicate motifs (FIG. 3). Structurally, the methylated ODNA (5′-CAG/iMe-dC/GGCGCCG/iMedC/GGCGCCTG) contains two 5-methylated cytosines. The last three bases at both the 5′- and 3′-ends have a nuclease-resistant phosphorothioate backbone. The unmethylated ODNB or the other ODN(X) shares an identical sequence with ODNA, containing the same phosphorothioate modifications at both ends. The in vivo evaluation conducted in a murine model demonstrates that conjugating methylated CpG moieties with ovalbumin allergen (OVA+ODNA) inhibits inflammation by stimulating IL-10, a key cytokine associated with Treg cells, while also reducing mast cell activity (FIG. 4). In contrast, unmethylated CpG moieties (OVA+ODNB) promote inflammatory cytokines through the Th2/Th17 pathways, resulting in increased levels of IL-4, IL-5, and IL-6 cytokines. These molecular effects directly correlate with IgE responses in this murine model of allergic disease, with the methylated ODNA moiety alleviating allergic symptoms and associated cytokines, while the ODNB moiety exacerbates them.

The compound to be administered will promote the recruitment of Treg cells in the patient, which will in turn regulate the immune system dysfunction and prevent the immune system from responding so aggressively that the host begins to suffer severe tissue damage from the deleterious actions of the host's immune system.

A patient can receive the treatment as a series of (subcutaneous or sublingual or intramuscular) injections or by oral ingestion or inhalation or by topical dermatological application over a fixed period of time, such that the population of Treg cells produced will provide immediate relief from the effects of an overactive immune system.

### Improvements on Pre-Existing Inventions

A method proposed by Oliver Lawless (U.S. Pat. No. 7,884,196) (see also Reference #1) employs compounds similar to these, derived from calf thymus. Since the genetic composition of these compounds is similar to that of mammalian cell DNA, including human cells, the methods proposed by Lawless present the somewhat ironic challenge of threatening patients with allergic reactions unrelated to the original condition for which the patient seeks treatment. Therefore, once the appropriate techniques are mastered, it is preferable to use a DNA source that is significantly more distantly related to the human genome for the proposed vaccine.

Our source, by contrast, is synthetic methylated DNA oligodeoxynucleotide (ODNA) specifically designed based on sequences derived from the B1-T2 m5C motif of B. longum subsp. infantis described in our previous publication (Reference #2). Bifidobacterium longum subsp. infantis bacterium is a bacterium found in the human gut, which we expect to be much more benign—and therefore much more widely applicable—due to the bacterium's long natural symbiosis with humans and its status as a ‘friendly’ commensal bacterium.

### Potential Adverse Side Effects Associated With the Proposed Treatment

While the stimulation of regulatory T cells (Tregs) shows promise in treating allergic and autoimmune diseases where Treg numbers are diminished, there are potential adverse effects to consider. In particular, excessive or non-specific Treg activation may inadvertently suppress beneficial immune responses, especially in the context of tumor immunity, where Tregs can inhibit anti-tumor responses and potentially promote tumor progression.

To address these concerns, we have designed strategies to minimize the risks associated with generalized Treg stimulation. Instead of inducing broad, nonspecific Treg activation, we employ a more targeted approach. By coupling specific antigens with CpG motifs, we have demonstrated a capability to induce a more selective and localized Treg response. In our recent in vivo mouse study (Reference #3). We show that by conjugating ovalbumin (OVA) with CpG moieties, we can restrict Treg activation to antigen-specific immune responses, thereby avoiding widespread immunosuppression.

Our findings suggest that this targeted approach effectively modulates the balance between Th1 and Th2 immune responses without significantly increasing the risk of autoimmunity or tumor growth. This method provides a more refined therapeutic strategy, reducing potential adverse side effects while maximizing the benefits of Treg stimulation in allergic and autoimmune conditions.

### Other Uses or Applications for This Invention

The compound is a natural target for further investigation in controlling a wide range of diseases, particularly the allergic and autoimmune disorders. For example, it may also become a key component of a new generation of anti-rejection therapies that could not only be useful in treating a variety of other immunologically-mediated diseases such as ‘long COVID’ but also beneficial in preventing graft rejection in human transplantation.

Another potential application of this compound is as a predictive marker for assessing the efficacy of specific probiotic preparation for patients. Our invention may significantly impact the probiotic industry, particularly because, among the thousands of probiotic products available for restoring health, there are currently no objective criteria to evaluate their efficacy. By utilizing our finding that the BI-T2 m5C motif of B. longum subsp. infantis (Reference #3) can stimulate Treg cells, this motif could serve as a valuable biomarker for assessing the effectiveness of probiotic products. This would provide a reliable measure of efficacy that is currently unavailable with other companies'offerings. Such an advancement would allow for more tailored probiotic preparations, enhancing patient care.

One potential avenue for enhancing the effectiveness of this compound is to reduce the risk of potential side effects associated with inducing generalized immunosuppression. This reduction can help avoid unintended consequences such as increased susceptibility to infections, autoimmune reactions, or cancer. One strategy that has been explored in the development of therapeutic vaccines to obviate these adverse effects is through the conjugation of CpG with a specific antigen. This procedure is known to shift generalized immunosuppression towards a more limited antigen-specific immunosuppression.

It may even prove practicable, for certain patients, to administer a schedule of applications of certain amounts of the methylated motifs along with certain amounts of the unmethylated variants of the same motifs, thereby provoking but also modulating the patient's immune response.

While we anticipate that the primary route of administration for the compound described in this application will be injection, other delivery methods-such as inhalation, oral ingestion, or topical application to the skin and mucosal surfaces, including the nasal mucosa-may also be effective and safe, and are not excluded from this disclosure. For example, administration by nasal spray may be facilitated and the compound stabilized by the addition of a nuclease-resistant phosphorothioate backbone. Additionally, delivering the compound as a nasal spray could enhance absorption for treating neurodegenerative disorders like Alzheimer's disease, due to the close proximity of the nasal cavity to the central nervous system.

We claim:

1. A vaccine composition comprising a motif (M) present in a human commensal bacterium, the motif possesses the capacity for Treg induction, wherein the motif (M) may undergo a bio-engineered process or a chemical process in order to achieve said capacity.

2. A vaccine regimen for allergic or autoimmune or other disorders, the vaccine regimen comprising the administration of the vaccine composition of claim 1, the regimen further comprising one or more applications of the vaccine composition by means of injection or inhalation or topically or by mouth at fixed intervals over a first period of time, the vaccine then providing protection or relief for patients from the effects of the allergic or autoimmune disorders over a second period of time.

3. A method for predicting the effectiveness or efficacy of a probiotic preparation, the method employing the vaccine composition of claim 1.

4. A method for avoiding certain side effects arising from the use of the vaccine composition of claim 1, the method comprising conjugating one or more of the motifs of the vaccine composition of claim 1 with one or more known antigens in order to provoke a tailored response in a host's immune system.

5. The vaccine composition of claim 1, wherein the motif (M) is one of a cytosine-phosphodiester-guanine (CpG) motif and a cytosine-phosphorothioate-guanine (CPSG) motif.

6. The vaccine composition of claim 1, wherein the motif (M) is a processed motif rather than an unprocessed, or naive, motif.

7. The vaccine composition of claim 6, wherein the process applied is a sequence of one or more of methylation, ethylation, propylation, acetylation, formylation, acetylation, acylation, ketonization, phosphorylation, hydration, and hydroxylation.

8. The vaccine composition of claim 7, wherein the motif (M) comprises one or more occurrences of one or more of the BI-T2 m5C motifs (RGC*GGCGCC), (RGCGGC*GCC), and (RGC*GGC*GCC), and “C*” represents a motif processed according to one or more of the processes recited in claim 7.

9. The vaccine composition of clam 7, wherein the association of a specific antigen with the CpG moiety prevents the general suppression of a cytokine response in cancer patients, and substitutes a Treg response specifically targeted against a known specific antigen.

10. The vaccine regimen of claim 2, wherein the compound is stabilized by the addition of a nuclease-resistant phosphorothioate backbone.

11. The vaccine composition of claim 1, wherein the commensal organism is one or more strains of any or one or more of the Bifidobacterium bacterium, the Lactobacillus bacterium, and the Saccharomyces yeast.
